(19) United States
(12) Patent Application Publication
(10) Pub. No.: US 2019/0270659 A1
(43) Pub. Date: Sep. 5, 2019

(54) BIOCARRIER

(71) Applicant: YEN-PO CHEN, Taichung City (TW)

(72) Inventor: YEN-PO CHEN, Taichung City (TW)

(21) Appl. No.: 16/346,428

(22) PCT Filed: Nov. 10, 2016
(86) PCT No.: PCT/CN2016/105287

§ 371 Date: Apr. 30, 2019

(51) Int. Cl. C02F 3/10; C12M 3/00

(52) U.S. Cl. CPC C02F 3/109; C12M 23/48; C02F 3/108

(57) ABSTRACT

A biocarrier, including: a base plate, having a first face and a second face opposite to each other, the base plate further having two lateral sides, each of the lateral sides being located between the first face and the second face, a width direction being defined by a direction along which the two lateral side are aligned; a plurality of fin plates, the fin plates being spacedly arranged on the first face and the second face along the width direction, a gap being formed between any two adjacent ones of the fin plates; two protection plates, arranged at the two lateral sides of the base plate respectively, a face of each of the protection plates opposite to the base plate having a convex arc face, a width of each of the protection plates parallel to the base plate being larger than each of the gaps.

### BACKGROUND OF THE INVENTION

### Field of the Invention

The present invention relates to a biocarrier.

### Description of the Prior Art

Contaminant in waste water includes inorganic contaminant and organic contaminant. Inorganic contaminant includes ionic contaminant, such as copper, chromium, arsenic, and fluorine, and particulate matters generated from the process of grinding and cutting. Inorganic contaminant is usually removed from waste water by chemical coagulation. On the other hand, most of organic contaminant, such as alcohol, ketone, and carbohydrate, and some inorganic contaminant from metabolism of microorganism, such as ammonia, nitrate, and phosphate, are usually processed by microorganism. Microorganism acquires energy for growing and material for synthesizing cells. Contaminant in waste water can be removed by the metabolism and synthesis of microorganism.

The bio-method includes the suspended-type bioreactor and biofilm-type bioreactor. The suspended-type bioreactor has microorganism floc suspended in the reactor, such as MBR and activation sludge aeration tank. The biofilm-type bioreactor has a fixed surface for microorganism to adhere onto and grow. The microorganism forms a biofilm on the surface. The biofilm-type bioreactor includes contact aeration tank and MBBR, MBBR is becoming common recently. It has biocarriers in flowing water to provide growing surface to microorganism to loon the biofilm. Thus, much more microorganism can be grown in a small tank. In addition, the biofilm contacts the waste water sufficiently so the size of tank can be minimized. MBBR is suitable for small factories to reduce cost and acquire better performance.

Normal biocarrier is a base plate with plural holes for biofilm to adhere onto. However, water flow easily destroys the biofilm in the holes and the surface area of the biofilm is small to have a bad efficiency.

U.S. Pat. No. 6,036,863 shows that the base plate with plural fin plates protruded from the base plate. The fin plates help increase the surface area of the biofilm. However, the fin plates are exposed, when plural biocarriers are stacked in the tank, the fin plates may insert into the gap between the fin plates of another biocarrier to destroy the biofilm.

### SUMMARY OF THE INVENTION

The main object of the present invention is to provide a biocarrier which increase the area of biofilm and to prevent the biofilin from being damaged.

To achieve the above and other objects, the biocarrier of the present invention includes a base plate, a plurality of fin plates, and two protection plates.

The base plate has a first face and a second face opposite to each other. The base plate further has two lateral sides. Each of the lateral sides is located between the first face and the second face. A width direction is defined by a direction along which the two lateral side are aligned. The plurality of fin plates are spacedly arranged on the first face and the second face along the width direction. A gap is formed between any two adjacent ones of the fin plates. The two protection plates are arranged at the two lateral sides of the base plate respectively. A face of each of the protection plates opposite to the base plate has a convex arc face. A width of each of the protection plates parallel to the base plate is larger than each of the gaps.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of the present invention;

FIG. 2 is a stereogram showing a second embodiment of the present invention;

FIG. 3 is a top view of the present invention;

FIG. 4 is a top showing a second embodiment of the present invention;

FIG. 5 is a top showing a third embodiment of the present invention;

FIG. 6 is a top showing a fourth embodiment of the present invention;

FIG. 7 is a top showing a fifth embodiment of the present invention;

FIG. 8 is a top showing a sixth embodiment of the present invention;

FIG. 9 is an illustration of using of the present invention.

### DETAILED DESCRIPTION OF THE PREFERRED EMBODIMENTS

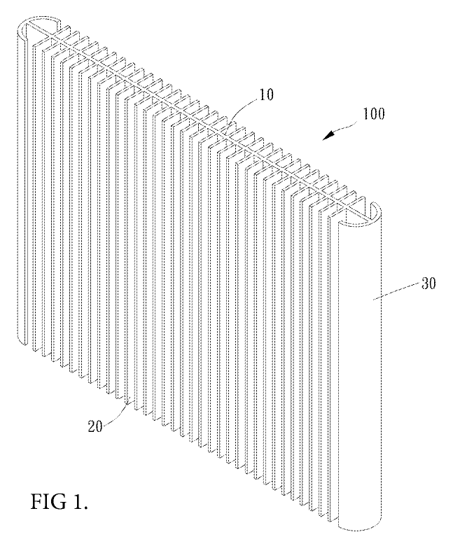

Please refer to FIG. 1 to FIG. 8, the biocarrier 100,100a,100b,100c,100d,100e of the present invention includes a base plate 10,10a, a plurality of fin plates 20,20a,20b,20c and two protection plates 30.

The base plate 10,10a has a first face 11 and a second face 12 opposite to each other. The base plate 10,10a further has two opposite lateral sides. Each of the lateral sides is located between the first face 11 and the second face 12. A width direction is defined as a direction along which the two lateral sides are aligned. The fin plates 20,20a,20b,20c are spacedly arranged on the first face 11 and the second face 12. A gap 40 is formed between any two adjacent ones of the fin plates 20,20a,20b,20c. A first height is defined as a distance from an end of each of the fin plates 20,20a,20b,20c remote from the base plate 10,10a to the base plate 10,10a. The two protection plates 30 are arranged at the two lateral sides of the base plate 10,10a respectively. A face of each of the protection plates 30 opposite to the base plate 10,10a has a convex arc face. A second height is defined as a distance from an end of each of the protection plates 30 remote from the base plate 10,10a to the base plate 10,10a. The first height is smaller than or equal to the second height. A width of each of the protection plates 30 parallel to the base plate 10 is larger than a width of each of the gaps 40. Preferably, each of the protection plates 30 has a C-shaped cross-section. Each of the protection plates 30 is connected to the lateral sides of the base plate 10,10a by an intermediate portion thereof. The base plate 10,10a, the fin plates 20,201,20b,20c, and the protection plates 30 are formed integrally by plastic material, such as PP, PET or HDPE, and have a density of 0.9-1.2 gram per cubic centimeters. In other possible embodiments, each of the protection plates can be composed of at least three flat sections connected one by one to have a polygonal shape similar to C-shape.

Preferably, the thickness of the base plate 10,10a or each of the fin plates 20,20a,20b,20c is 0.2-2 mm. The width of each of the gaps 40 is 0.3-5 mm. The width of the base plate 10,10a is 145 cm. The second height is 1-50 mm.

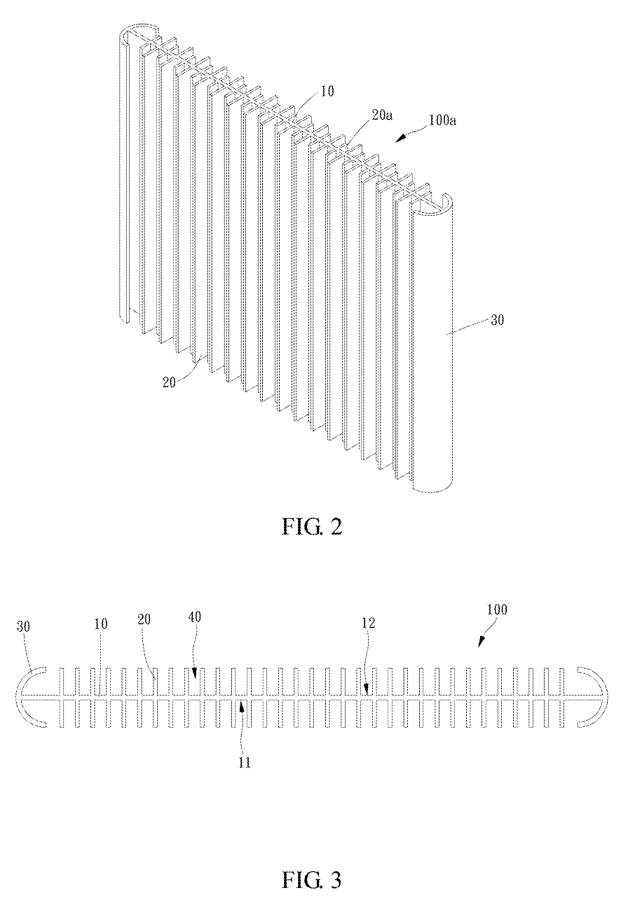

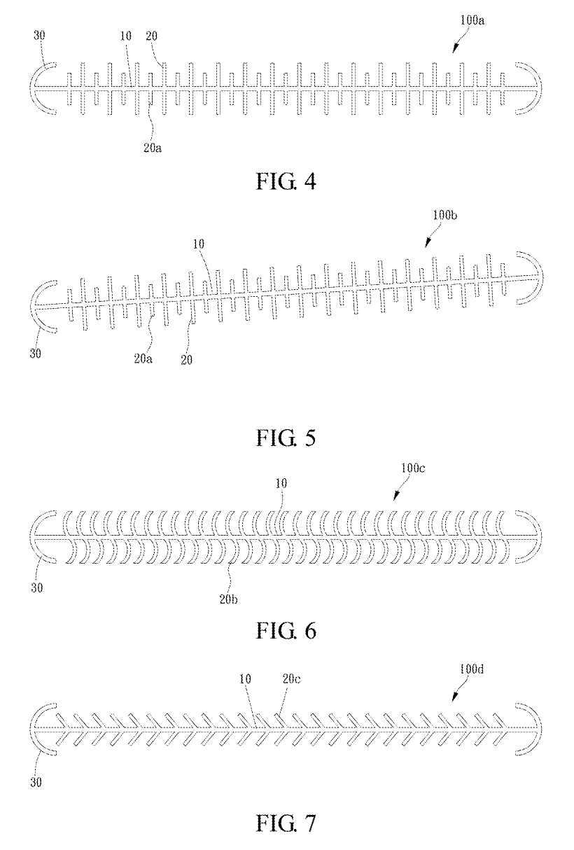

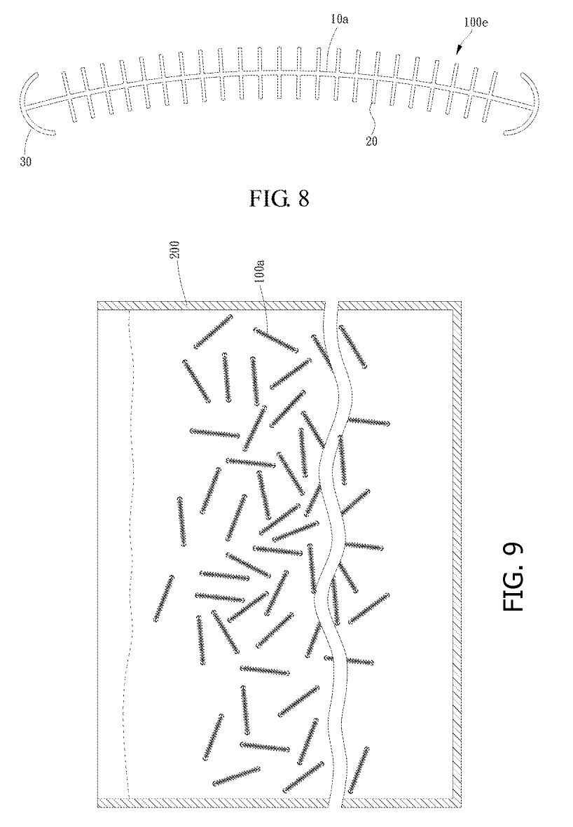

In the first embodiment of the present invention, as shown in FIG. 1 and FIG. 3 the fin plates 20 have the same length and are all perpendicular to the base plate 10. This structure is suitable for biofilm of anaerobic bacteria. In other possible embodiments, as shown in FIG. 2 and FIG. 4 the fin plates 20,20a include a plurality of long fin plates and a plurality of short fin plate& The first height of each of the long fin plates is larger than the first height of each of the short fin plates. The long fin plates and the short fin plates are arranged alternately. Thereby, the biofilm among the fin plates 20,20a has a concave arc shape to be suitable for the biofilm of aerobic bacteria. On the other hand, FIG. 5 shows that the protection plates 30 are not perpendicular to the base plate 10, and FIG. 6 shows that the fin plates 20b are arc-shaped. FIG. 7 further shows that the fin plates 20c on the first face and the fin plates 20c on the second face are inclined toward a same direction to be the shape of fish bone. In other words, an angle between each of the fin plates and the base plate is not 90 degrees. The fin plates on the first face are parallel to each other, and the fin plates on the second face are also parallel to each other. FIG. 8 further shows that the base plate 10a is arc-shaped.

In use, as shown in FIG. 9, the biocarrier 100a is placed in a container 200 to purify the water in the container. Specifically, (preferably plural) the biocarrier can be placed in a water-purifier. The fin plates are just like abutments of bridge. The biofilm of microorganism will form on the fin plates and among the fin plates. The biofilm can used to decompose the toxin and purify the water. In addition, to prevent the biocarriers rub against each other, the longer protection plates help protect the fin plates. Besides the convex arc-shaped outer surface of the protection plate can avoid the terminal end of the protection plates or the fin plates of other biocarriers from inserting into the gaps to destroy the biofilm.

Besides, the gaps between the fin plates are protected by the fin plates so as to slow down the water flow and to decrease the shear force of aeration. Thus, the biofilm is easier to form and maintains sufficient thickness to be avoided from peeling off. When the biocarrier of the present invention is used in the processing of organic waste water, the anaerobic microorganism layer helps increase the organic load, decrease the aeration, and decrease the biological sludge. When the biocarrier of the present invention is used in processing of nitrogen waste water, the aerobic microorganism layer helps increase nitrogen load and decrease the cost.

What is claimed is:

1. A biocarrier, including:
a base plate, having a first face and a second face opposite to each other, the base plate further having two lateral sides, each of the lateral sides being located between the first face and the second face, a width direction being defined by a direction along which the two lateral side are aligned;
a plurality of fin plates, the plurality of fin plates being spacedly arranged on the first face and the second face along the width direction, a gap being formed between any two adjacent ones of the plurality of fin plates;
two protection plates, arranged at the two lateral sides of the base plate respectively, a face of each of the protection plates opposite to the base plate having a convex arc face, a width of each of the protection plates parallel to the base plate being larger than each of the gaps.

2. The biocarrier of claim 1, wherein the base plate is a flat, plate or an arc plate.

3. The biocarrier of claim 1, wherein a first height is defined as a distance from an end of each of the fin plates remote from the base plate to the base plate, a second height is defined as a distance from an end of each of the protection plates remote from the base plate to the base plate, the first height is smaller than or equal to the second height.

4. The biocarrier of claim 1, wherein each of the fin plates is perpendicular to the base plate.

5. The biocarrier of claim 1, wherein an angel between each of the fin plates and the base plate is smaller than or larger than 90 degrees, the fin plates on the first face of the base plate are parallel to each other, the fin plates on the second face of the base plate are parallel to each other.

6. The biocarrier of claim 3, wherein the fin plates includes a plurality of long fin plates and a plurality of short fin plates, the first height of each of the long fin plates is larger than the first height of each of the short fin plates, the long fin plates and the short fin plates are arranged alternately.

7. The biocarrier of claim 1, wherein each of the protection plate has a C-shaped cross-section, each of the protection plates is connected to one of the lateral sides of the base plate by an intermediate portion thereof.

8. The biocarrier of claim 1, wherein each of the protection plates includes at least three flat sections connected one by one to have a polygonal shape.

9. The biocarrier of claim 1, wherein the base plate, the fin plates, and the protection plates are formed integrally by plastic material.

10. The biocarrier of claim 9, wherein the biocarrier has a density of 0.9-1.2 g/cubic centimeters.
